SEQUENCE LISTING (4 sequences)

SEQ ID NO: 1 - DNA, 83 bp, synthetic construct
  misc_feature 1..83: note = Synthetic Polynucleotide
  source 1..83: mol_type = other DNA
actagtattatgcccagtacatgaccttatgggactttcctacttggcagtacatctacgtattagtcatcgctattaccatg

SEQ ID NO: 2 - DNA, 350 bp, synthetic construct
  misc_feature 1..350: note = Synthetic Polynucleotide
  source 1..350: mol_type = other DNA
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaaga

SEQ ID NO: 3 - DNA, 5411 bp, synthetic construct
  misc_feature 1..5411: note = Synthetic Polynucleotide
  source 1..5411: mol_type = other DNA
ggctccccgggcgcgactagtgaattgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagtcgacgccaccatgggcaacttgaagagcgtggcccaggagcctgggccaccctgcggcctggggctggggctgggccttgggctgtgcggcaagcagggcccagccaccccggcccctgagcccagccgggccccagcatccctactcccaccagcgccagaacacagccccccgagctccccgctaacccagcccccagaggggcccaagttccctcgtgtgaagaactgggaggtggggagcatcacctatgacaccctcagcgcccaggcgcagcaggatgggccctgcaccccaagacgctgcctgggctccctggtatttccacggaaactacagggccggccctcccccggccccccggcccctgagcagctgctgagtcaggcccgggacttcatcaaccagtactacagctccattaagaggagcggctcccaggcccacgaacagcggcttcaagaggtggaagccgaggtggcagccacaggcacctaccagcttagggagagcgagctggtgttcggggctaagcaggcctggcgcaacgctccccgctgcgtgggccggatccagtgggggaagctgcaggtgttcgatgcccgggactgcaggtctgcacaggaaatgttcacctacatctgcaaccacatcaagtatgccaccaaccggggcaaccttcgctcggccatcacagtgttcccgcagcgctgccctggccgaggagacttccgaatctggaacagccagctggtgcgctacgcgggctaccggcagcaggacggctctgtgcggggggacccagccaacgtggagatcaccgagctctgcattcagcacggctggaccccaggaaacggtcgcttcgacgtgctgcccctgctgctgcaggccccagatgagcccccagaactcttccttctgccccccgagctggtccttgaggtgcccctggagcaccccacgctggagtggtttgcagccctgggcctgcgctggtacgccctcccggcagtgtccaacatgctgctggaaattgggggcctggagttccccgcagcccccttcagtggctggtacatgagcactgagatcggcacgaggaacctgtgtgaccctcaccgctacaacatcctggaggatgtggctgtctgcatggacctggatacccggaccacctcgtccctgtggaaagacaaggcagcagtggaaatcaacgtggccgtgctgcacagttaccagctagccaaagtcaccatcgtggaccaccacgccgccacggcctctttcatgaagcacctggagaatgagcagaaggccagggggggctgccctgcagactgggcctggatcgtgccccccatctcgggcagcctcactcctgttttccatcaggagatggtcaactatttcctgtccccggccttccgctaccagccagacccctggaaggggagtgccgccaagggcaccggcatcaccaggaagaagacctttaaagaagtggccaacgccgtgaagatctccgcctcgctcatgggcacggtgatggcgaagcgagtgaaggcgacaatcctgtatggctccgagaccggccgggcccagagctacgcacagcagctggggagactcttccggaaggcttttgatccccgggtcctgtgtatggatgagtatgacgtggtgtccctcgaacacgagacgctggtgctggtggtaaccagcacatttgggaatggggatcccccggagaatggagagagctttgcagctgccctgatggagatgtccggcccctacaacagctcccctcggccggaacagcacaagagttataagatccgcttcaacagcatctcctgctcagacccactggtgtcctcttggcggcggaagaggaaggagtccagtaacacagacagtgcaggggccctgggcaccctcaggttctgtgtgttcgggctcggctcccgggcatacccccacttctgcgcctttgctcgtgccgtggacacacggctggaggaactgggcggggagcggctgctgcagctgggccagggcgacgagctgtgcggccaggaggaggccttccgaggctgggcccaggctgccttccaggccgcctgtgagaccttctgtgtgggagaggatgccaaggccgccgcccgagacatcttcagccccaaacggagctggaagcgccagaggtaccggctgagcgcccaggccgagggcctgcagttgctgccaggtctgatccacgtgcacaggcggaagatgttccaggctacaatccgctcagtggaaaacctgcaaagcagcaagtccacgagggccaccatcctggtgcgcctggacaccggaggccaggaggggctgcagtaccagccgggggaccacataggtgtctgcccgcccaaccggcccggccttgtggaggcgctgctgagccgcgtggaggacccgccggcgcccactgagcccgtggcagtagagcagctggagaagggcagccctggtggccctccccccggctgggtgcgggacccccggctgcccccgtgcacgctgcgccaggctctcaccttcttcctggacatcacctccccacccagccctcagctcttgcggctgctcagcaccttggcagaagagcccagggaacagcaggagctggaggccctcagccaggatccccgacgctacgaggagtggaagtggttccgctgccccacgctgctggaggtgctggagcagttcccgtcggtggcgctgcctgccccactgctcctcacccagctgcctctgctccagccccggtactactcagtcagctcggcacccagcacccacccaggagagatccacctcactgtagctgtgctggcatacaggactcaggatgggctgggccccctgcactatggagtctgctccacgtggctaagccagctcaagcccggagaccctgtgccctgcttcatccggggggctccctccttccggctgccacccgatcccagcttgccctgcatcctggtgggtccaggcactggcattgcccccttccggggattctggcaggagcggctgcatgacattgagagcaaagggctgcagcccactcccatgactttggtgttcggctgccgatgctcccaacttgaccatctctaccgcgacgaggtgcagaacgcccagcagcgcggggtgtttggccgagtcctcaccgccttctcccgggaacctgacaaccccaagacctacgtgcaggacatcctgaggacggagctggctgcggaggtgcaccgcgtgctgtgcctcgagcggggccacatgtttgtctgcggcgatgttaccatggcaaccaacgtcctgcagaccgtgcagcgcatcctggcgacggagggcgacatggagctggacgaggccggcgacgtcatcggcgtgctgcgggatcagcaacgctaccacgaagacattttcgggctcacgctgcgcacccaggaggtgacaagccgcatacgcacccagagcttttccttgcaggagcgtcagttgcggggcgcagtgccctgggcgttcgaccctcccggctcagacaccaacagcccctgagagccgcctggctttcccttccagttccgggagagcggctgcccgactcaggtccgcccgaccaggggaattcgctagctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagatcgacccatgggggcccgccccaactggggtaacctttg

SEQ ID NO: 4 - DNA, 6034 bp, synthetic construct
  misc_feature 1..6034: note = Synthetic Polynucleotide
  source 1..6034: mol_type = other DNA
ggctccccgggcgcgactagtgaattgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagtcgacgccaccatgggcaacttgaagagcgtggcccaggagcctgggccaccctgcggcctggggctggggctgggccttgggctgtgcggcaagcagggcccagccaccccggcccctgagcccagccgggccccagcatccctactcccaccagcgccagaacacagccccccgagctccccgctaacccagcccccagaggggcccaagttccctcgtgtgaagaactgggaggtggggagcatcacctatgacaccctcagcgcccaggcgcagcaggatgggccctgcaccccaagacgctgcctgggctccctggtatttccacggaaactacagggccggccctcccccggccccccggcccctgagcagctgctgagtcaggcccgggacttcatcaaccagtactacagctccattaagaggagcggctcccaggcccacgaacagcggcttcaagaggtggaagccgaggtggcagccacaggcacctaccagcttagggagagcgagctggtgttcggggctaagcaggcctggcgcaacgctccccgctgcgtgggccggatccagtgggggaagctgcaggtgttcgatgcccgggactgcaggtctgcacaggaaatgttcacctacatctgcaaccacatcaagtatgccaccaaccggggcaaccttcgctcggccatcacagtgttcccgcagcgctgccctggccgaggagacttccgaatctggaacagccagctggtgcgctacgcgggctaccggcagcaggacggctctgtgcggggggacccagccaacgtggagatcaccgagctctgcattcagcacggctggaccccaggaaacggtcgcttcgacgtgctgcccctgctgctgcaggccccagatgagcccccagaactcttccttctgccccccgagctggtccttgaggtgcccctggagcaccccacgctggagtggtttgcagccctgggcctgcgctggtacgccctcccggcagtgtccaacatgctgctggaaattgggggcctggagttccccgcagcccccttcagtggctggtacatgagcactgagatcggcacgaggaacctgtgtgaccctcaccgctacaacatcctggaggatgtggctgtctgcatggacctggatacccggaccacctcgtccctgtggaaagacaaggcagcagtggaaatcaacgtggccgtgctgcacagttaccagctagccaaagtcaccatcgtggaccaccacgccgccacggcctctttcatgaagcacctggagaatgagcagaaggccagggggggctgccctgcagactgggcctggatcgtgccccccatctcgggcagcctcactcctgttttccatcaggagatggtcaactatttcctgtccccggccttccgctaccagccagacccctggaaggggagtgccgccaagggcaccggcatcaccaggaagaagacctttaaagaagtggccaacgccgtgaagatctccgcctcgctcatgggcacggtgatggcgaagcgagtgaaggcgacaatcctgtatggctccgagaccggccgggcccagagctacgcacagcagctggggagactcttccggaaggcttttgatccccgggtcctgtgtatggatgagtatgacgtggtgtccctcgaacacgagacgctggtgctggtggtaaccagcacatttgggaatggggatcccccggagaatggagagagctttgcagctgccctgatggagatgtccggcccctacaacagctcccctcggccggaacagcacaagagttataagatccgcttcaacagcatctcctgctcagacccactggtgtcctcttggcggcggaagaggaaggagtccagtaacacagacagtgcaggggccctgggcaccctcaggttctgtgtgttcgggctcggctcccgggcatacccccacttctgcgcctttgctcgtgccgtggacacacggctggaggaactgggcggggagcggctgctgcagctgggccagggcgacgagctgtgcggccaggaggaggccttccgaggctgggcccaggctgccttccaggccgcctgtgagaccttctgtgtgggagaggatgccaaggccgccgcccgagacatcttcagccccaaacggagctggaagcgccagaggtaccggctgagcgcccaggccgagggcctgcagttgctgccaggtctgatccacgtgcacaggcggaagatgttccaggctacaatccgctcagtggaaaacctgcaaagcagcaagtccacgagggccaccatcctggtgcgcctggacaccggaggccaggaggggctgcagtaccagccgggggaccacataggtgtctgcccgcccaaccggcccggccttgtggaggcgctgctgagccgcgtggaggacccgccggcgcccactgagcccgtggcagtagagcagctggagaagggcagccctggtggccctccccccggctgggtgcgggacccccggctgcccccgtgcacgctgcgccaggctctcaccttcttcctggacatcacctccccacccagccctcagctcttgcggctgctcagcaccttggcagaagagcccagggaacagcaggagctggaggccctcagccaggatccccgacgctacgaggagtggaagtggttccgctgccccacgctgctggaggtgctggagcagttcccgtcggtggcgctgcctgccccactgctcctcacccagctgcctctgctccagccccggtactactcagtcagctcggcacccagcacccacccaggagagatccacctcactgtagctgtgctggcatacaggactcaggatgggctgggccccctgcactatggagtctgctccacgtggctaagccagctcaagcccggagaccctgtgccctgcttcatccggggggctccctccttccggctgccacccgatcccagcttgccctgcatcctggtgggtccaggcactggcattgcccccttccggggattctggcaggagcggctgcatgacattgagagcaaagggctgcagcccactcccatgactttggtgttcggctgccgatgctcccaacttgaccatctctaccgcgacgaggtgcagaacgcccagcagcgcggggtgtttggccgagtcctcaccgccttctcccgggaacctgacaaccccaagacctacgtgcaggacatcctgaggacggagctggctgcggaggtgcaccgcgtgctgtgcctcgagcggggccacatgtttgtctgcggcgatgttaccatggcaaccaacgtcctgcagaccgtgcagcgcatcctggcgacggagggcgacatggagctggacgaggccggcgacgtcatcggcgtgctgcgggatcagcaacgctaccacgaagacattttcgggctcacgctgcgcacccaggaggtgacaagccgcatacgcacccagagcttttccttgcaggagcgtcagttgcggggcgcagtgccctgggcgttcgaccctcccggctcagacaccaacagcccctgagagccgcctggctttcccttccagttccgggagagcggctgcccgactcaggtccgcccgaccaggggaattcgctagctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttctagagggcccgagatctttttccctctgccaaaaattatggggacatcatgaagccccttgagcatctgacttctggctaataaaggaaatttattttcattgcaatagtgtgttggaattttttgtgtctctcactcggaaggacataagggcggccctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagatcgacccatgggggcccgccccaactggggtaacctttg

(19) United States
(12) Patent Application Publication
(10) Pub. No.: US 2026/0151430 A1
(43) Pub. Date: Jun. 4, 2026

(54) Constructs for Enhanced Production of Endothelial Nitric Oxide Synthase and Methods of Producing Cellular Compositions for Treatment of Pulmonary and Cardiac Diseases

(71) Applicant: Ottawa Hospital Research Institute, Ottawa (CA)

(72) Inventors: Duncan Stewart, Ottawa (CA); David Courtman, Ottawa (CA)

(73) Assignee: Ottawa Hospital Research Institute, Ottawa, ON (CA)

(21) Appl. No.: 18/686,120

(22) PCT Filed: Aug. 22, 2022
(86) PCT No.: PCT/CA2022/051267

§ 371 Date: Feb. 23, 2024

Related U.S. Application Data
(60) Provisional application No. 63/237,027, filed on Aug. 25, 2021.

(51) Int. Cl. A61K 35/28; A61K 38/44; A61P 9/10; A61P 9/12; C12N 5/0775; C12N 15/85

(52) U.S. Cl. CPC A61K 35/28; A61K 38/44; A61P 9/10; A61P 9/12; C12N 5/0663; C12N 15/85; C12Y 114/13039; C12N 2501/105; C12N 2501/115; C12N 2501/165; C12N 2501/39; C12N 2533/52; C12N 2800/107

(57) ABSTRACT

Nucleic acid molecules comprising truncated forms of the human cytomegalovirus (CMV) promoter are operably linked to a transgene of interest, including those encoding eNOS protein are taught. Vectors comprising these nucleic acid molecules and host cells transformed by these vectors and methods of producing cellular compositions are used for the treatment of a variety of pulmonary and cardiac diseases. There is provided a truncated human cytomegalovirus (CMV) enhancer element comprising SEQ ID NO: 1 or a functional derivative thereof. A truncated human cytomegalovirus (CMV) promoter comprising the truncated CMV enhancer element is taught. A polynucleotide expression cassette comprising the truncated CMV promoter and a transcribable polynucleotide operably linked to the truncated CMV promoter polynucleotide construct, a host cell comprising the polynucleotide expression cassette, and use of the host cell treat renal, vascular, pulmonary, or cardiac disease in the patient are taught.

### TECHNICAL FIELD

The present disclosure relates to nucleic acid molecules comprising truncated forms of the human cytomegalovirus (CMV) promoter operably linked to a transgene of interest, including those encoding eNOS protein. This disclosure further relates to vectors comprising these nucleic acid molecules and host cells transformed by these vectors and methods of producing cellular compositions for the treatment of a variety of pulmonary and cardiac diseases.

### BACKGROUND

In cell-based gene transfer, DNA sequences containing the genes which one desires to introduce into the patient's body (the transgenes) are prepared extracellularly, e.g. by using enzymatic cleavage and subsequent recombination of DNA with insert DNA sequences.

The insert gene is transferred to patient by culturing cells from the patient's own (i.e. autologous) or cells from another individual (i.e. allogenic) cells are then cultured in vitro and treated so as to take up the transgene in an expressible form. The transgenes may be foreign to the mammalian cell, or comprise additional copies of genes already present in the cell to increase the amount of expression product of the gene or copies of normal genes which may be defective or missing in a particular patient.

The take-up of the foreign gene by the cells in culture may be accomplished by genetic engineering techniques, e.g. by causing transfection of the cells with a plasmid vector containing the DNA of the gene to be transferred by lipofection, by electroporation, transfection with cationic polymers (e.g. natural or synthetic cationic polymers such as polyethylenimine or linear polyethylenimine) or by other accepted means to obtain transfected cells.

Once the cells have been transfected, it is sometimes beneficial to selectively culture the cells which have successfully taken up the transgene in an expressible form, so that administration of the cells to the patient can be limited to the transfected cells expressing the transgene. In other cases, all of the cells subject to the take-up process are administered.

The cells containing the transgene are introduced into the patient, so that the gene may express the required gene products in the body, for therapeutic purposes.

Current gene transfer vectors have several drawbacks when they are used for gene therapy purposes. Viral and bacterial-derived gene transfer vectors can induce the patient's innate and adaptive immune responses. For example, plasmid DNA (pDNA) vectors typically have a bacterial backbone sequences (e.g. ori, antibiotic resistance marker) or a prokaryotic pattern of DNA methylation that is not present in eukaryotic DNA.

One attractive method of introducing genetically altered cells or regenerative cells into the body, for purposes of gene therapy, is to use the pulmonary system. The pulmonary system has a number of unique features rendering it particularly suited to a cell-based gene transfer. Low arterial pressure and high surface area with relatively low shear in the micro-circulation of the lungs increase the chances of survival of the transplanted cells. High oxygenation in the micro-circulation of the ventilated lung also improves the viability of the transplanted cells.

Moreover, the pulmonary circulation functions as a natural filter, and is able to retain the infused cells efficiently and effectively. Also, the lung has a dual circulation (pulmonary arterial and bronchial). This is in contra-distinction to other systemic circulations, such as the brain and the heart, where the infusion of particulate materials such as cells could lead to the aforementioned adverse consequences. The lung presents a massive vascular system. The high surface area of the pulmonary endothelium allows the migration of the transplanted cells trapped in the micro-circulation across the endothelial layer to take up residence within the perivascular space.

The pulmonary circulation, unlike any other circulation in the body, receives the entire output of the heart. Accordingly, the pulmonary circulation present a great opportunity to release a gene product into the circulation. This distinct property of the lung is particularly useful for pulmonary gene therapy and for the treatment of a systemic disorders, as well as pulmonary disorders.

Without being limited to any particular theory, it is believed that cells become lodged in the small artery-capillary transition regions of the pulmonary circulation system, following simple intravenous injection of the transfected or regenerative cells to the patient. Products administered intravenously move with the venous circulation to the right side of the heart and then to the lungs. Administered cells appear to lodge in the small arteriolar-capillary transition regions of the circulatory system of the lungs, and then transmigrate from the intraluminal to the perivascular space. From there transfected cells deliver expression products of the transgenes to the lungs, making the process to the present disclosure especially applicable to treatment of pulmonary disorders. Some factors, especially stable factors can be secreted to the general circulation for treatment of disorders of other body organs.

Each year in the United States, approximately 1.2 million people suffer a myocardial infarction (MI) with an expected mortality of 45% within 1 year. Despite substantial improvements in pharmacological and interventional therapy of acute MI and other cardiac diseases over the last decades, 5.7 million patients suffer from heart failure, facing a 1-year mortality of 22%.

While early revascularization is undoubtedly the cornerstone of modern therapy for acute MI, many patients cannot be revascularized within the optimal therapeutic time window or do not demonstrate the expected improvement in cardiac function following reperfusion.

Some preclinical studies have shown that the use of adult stem or progenitor cells can result in dramatic reduction in infarct scar and marked improvement in myocardial contractility in experimental infarct models.

Cell therapy treatment using bone marrow derived mononuclear cells (MNCs) in the post-MI setting has been shown to achieve its primary endpoint of improvement in global left ventricular function (LVEF), measured by left ventricular angiography, at 4 months. Although the actual improvement was rather modest, with only a 2.5% difference between the cell and placebo groups.

Reduced eNOS expression and nitric oxide production have been strongly implicated in endothelial dysfunction which has been reported in patients with cardiac risk factors and/or CAD.

While there have been improvements in the route of delivery of the desired transgene(s), there remains the problem of achieving significant therapeutic expression of therapeutic gene(s) within the cell and a need to improve the methods of harvesting, transforming, and growing the host cells for use in the treatment of pulmonary and cardiac diseases.

Thus, there is a need for gene-based delivery of therapeutic proteins for the treatment of disease that provides regulated, long-term expression of the protein, resulting in therapeutic efficacy. In particular, there is a need to provide compositions for gene-based delivery of therapeutic proteins for the treatment of pulmonary and cardiac disease in a patient in need of treatment thereof.

### SUMMARY OF THE INVENTION

The present disclosure provides an improved expression system for the regulated expression of an encoded protein or nucleic acid therapeutic factor for use in the treatment of disease, wherein therapeutic efficacy of the therapeutic factor can be maximized.

In particular, the present disclosure provides an improved regulated gene expression system, pharmaceutical compositions and methods thereof for treatment of renal, vacular, pulmonary, and cardioavascular disease, and methods for preparing the medicaments for treatment of renal, vacular, pulmonary, and cardioavascular disease. The encoded therapeutic factor can be a nucleic acid or protein that provides a therapeutic benefit to a subject having, or susceptible to, a disease. For example, such therapeutic benefit or activity includes, but is not limited to, the amelioration, modulation, diminution, stabilization, or prevention of a disease or a symptom of a disease.

It is an embodiment of the present disclosure to provide a truncated CMV enhancer element or functional variant thereof. In other nonlimiting embodiments there is provided a truncated CMV promoter with the truncated CMV enhancer element or functional variant thereof. In other nonlimiting embodiments, the truncated CMV promoter may be operably linked to a transcribable gene, so that transcription of the gene is regulated by the truncated CMV enhancer promoter of the present disclosure.

In particular nonlimiting embodiments, the truncated CMV enhancer promoter is operably linked to a transcribable gene encoding a therapeutic factor. In some embodiments, the therapeutic factor is endothelial nitric oxide synthase (eNOS).

In one non-limiting embodiment, there is provided a truncated CMV promoter with the truncated CMV enhancer element capable of promoting significant expression of eNOS in endothelial progentitor cells (EPCs) or endothelial-like progenitor cells.

Also provided are polynucleotide constructs comprising the nucleic acid molecules of the present disclosure. In particular nonlimiting embodiments, the polynucleotide constructs of the present disclosure may comprise without limitation nanoplasmids, minicircle DNA, plasmids (e.g mini intronic plasmid (MIP)), minimalistic immunologically defined gene expression (MIDGE) vectors, and dbDNA (Doggybone™ DNA).

Also provided by the present disclosure are cells transformed by the polynucleotide constructs described herein.

In particular nonlimiting embodiments, the truncated CMV enhancer element of the present disclosure has the following nucleic acid sequence ID NO. 1:

ACTAGTATTATGCCCAGTACATGACCTTATGGGACTTTCCTACTTGGCA
GTACATCTACGTATTAGTCATCGCTATTACCATG

In particular nonlimiting embodiments, the truncated CMV enhancer element is a functional fragment having a sequence identity of from 80%, 85%, 90%, or 95% sequence identity to the following nucleic acid sequence ID NO. 1.

In particular nonlimiting embodiments, there is provided a truncated CMV promoter with the truncated CMV enhancer element of the present disclosure having the following nucleic acid sequence ID NO. 2:

ACTAGTATTATGCCCAGTACATGACCTTATGGGACTTTCCTACTTGGCA
GTACATCTACGTATTAGTCATCGCTATTACCATGGTGATGCGGTTTTGG
CAGTACATCAATGGGCGTGGATAGCGGTTTGACTCACGGGGATTTCCAA
GTCTCCACCCCATTGACGTCAATGGGAGTTTGTTTTGGCACCAAAATCA
ACGGGACTTTCCAAAATGTCGTAACAACTCCGCCCCATTGACGCAAATG
GGGGGTAGGCGTGTACGGTGGGAGGTTTATATAAGCAGAGCTCGTTTAG
TGAACCGTCAGATCGCCTGGAGACGCCATCCACGCTGTTTTGACCTCCA
TAGAAGA

In particular nonlimiting embodiments, the cells are transformed using a minicircle comprising the truncated CMV enhancer promoter operably linked to endothelial nitric oxide synthase (eNOS) having the sequence ID NO. 3:

GGCTCCCCGGGCGCGACTAGTGAATTGATACTAGTATTATGCCCAGTAC
ATGACCTTATGGGACTTTCCTACTTGGCAGTACATCTACGTATTAGTCA
TCGCTATTACCATGGTGATGCGGTTTTGGCAGTACATCAATGGGCGTGG
ATAGCGGTTTGACTCACGGGGATTTCCAAGTCTCCACCCCATTGACGTC
AATGGGAGTTTGTTTTGGCACCAAAATCAACGGGACTTTCCAAAATGTC
GTAACAACTCCGCCCCATTGACGCAAATGGGCGGTAGGCGTGTACGGTG
GGAGGTTTATATAAGCAGAGCTCGTTTAGTGAACCGTCAGATCGCCTGG
AGACGCCATCCACGCTGTTTTGACCTCCATAGAAGATTCTAGAGTCGAC
GCCACCATGGGCAACTTGAAGAGCGTGGCCCAGGAGCCTGGGCCACCCT
GCGGCCTGGGGCTGGGGCTGGGCCTTGGGCTGTGCGGCAAGCAGGGCCC
AGCCACCCCGGCCCCTGAGCCCAGCCGGGCCCCAGCATCCCTACTCCCA
CCAGCGCCAGAACACAGCCCCCCGAGCTCCCCGCTAACCCAGCCCCCAG
AGGGGCCCAAGTTCCCTCGTGTGAAGAACTGGGAGGTGGGGAGCATCAC
CTATGACACCCTCAGCGCCCAGGCGCAGCAGGATGGGCCCTGCACCCCA
AGACGCTGCCTGGGCTCCCTGGTATTTCCACGGAAACTACAGGGCCGGC
CCTCCCCCGGCCCCCCGGCCCCTGAGCAGCTGCTGAGTCAGGCCCGGGA
CTTCATCAACCAGTACTACAGCTCCATTAAGAGGAGCGGCTCCCAGGCC
CACGAACAGCGGCTTCAAGAGGTGGAAGCCGAGGTGGCAGCCACAGGCA
CCTACCAGCTTAGGGAGAGCGAGCTGGTGTTCGGGGCTAAGCAGGCCTG
GCGCAACGCTCCCCGCTGCGTGGGCCGGATCCAGTGGGGGAAGCTGCAG
GTGTTCGATGCCCGGGACTGCAGGTCTGCACAGGAAATGTTCACCTACA
TCTGCAACCACATCAAGTATGCCACCAACCGGGGCAACCTTCGCTCGGC
CATCACAGTGTTCCCGCAGCGCTGCCCTGGCCGAGGAGACTTCCGAATC
TGGAACAGCCAGCTGGTGCGCTACGCGGGCTACCGGCAGCAGGACGGCT
CTGTGCGGGGGGACCCAGCCAACGTGGAGATCACCGAGCTCTGCATTCA
GCACGGCTGGACCCCAGGAAACGGTCGCTTCGACGTGCTGCCCCTGCTG
CTGCAGGCCCCAGATGAGCCCCCAGAACTCTTCCTTCTGCCCCCCGAGC
TGGTCCTTGAGGTGCCCCTGGAGCACCCCACGCTGGAGTGGTTTGCAGC
CCTGGGCCTGCGCTGGTACGCCCTCCCGGCAGTGTCCAACATGCTGCTG
GAAATTGGGGGCCTGGAGTTCCCCGCAGCCCCCTTCAGTGGCTGGTACA
TGAGCACTGAGATCGGCACGAGGAACCTGTGTGACCCTCACCGCTACAA
CATCCTGGAGGATGTGGCTGTCTGCATGGACCTGGATACCCGGACCACC
TCGTCCCTGTGGAAAGACAAGGCAGCAGTGGAAATCAACGTGGCCGTGC
TGCACAGTTACCAGCTAGCCAAAGTCACCATCGTGGACCACCACGCCGC
CACGGCCTCTTTCATGAAGCACCTGGAGAATGAGCAGAAGGCCAGGGGG
GGCTGCCCTGCAGACTGGGCCTGGATCGTGCCCCCCATCTCGGGCAGCC
TCACTCCTGTTTTCCATCAGGAGATGGTCAACTATTTCCTGTCCCCGGC
CTTCCGCTACCAGCCAGACCCCTGGAAGGGGAGTGCCGCCAAGGGCACC
GGCATCACCAGGAAGAAGACCTTTAAAGAAGTGGCCAACGCCGTGAAGA
TCTCCGCCTCGCTCATGGGCACGGTGATGGCGAAGCGAGTGAAGGCGAC
AATCCTGTATGGCTCCGAGACCGGCCGGGCCCAGAGCTACGCACAGCAG
CTGGGGAGACTCTTCCGGAAGGCTTTTGATCCCCGGGTCCTGTGTATGG
ATGAGTATGACGTGGTGTCCCTCGAACACGAGACGCTGGTGCTGGTGGT
AACCAGCACATTTGGGAATGGGGATCCCCCGGAGAATGGAGAGAGCTTT
GCAGCTGCCCTGATGGAGATGTCCGGCCCCTACAACAGCTCCCCTCGGC
CGGAACAGCACAAGAGTTATAAGATCCGCTTCAACAGCATCTCCTGCTC
AGACCCACTGGTGTCCTCTTGGCGGCGGAAGAGGAAGGAGTCCAGTAAC
ACAGACAGTGCAGGGGCCCTGGGCACCCTCAGGTTCTGTGTGTTCGGGC
TCGGCTCCCGGGCATACCCCCACTTCTGCGCCTTTGCTCGTGCCGTGGA
CACACGGCTGGAGGAACTGGGCGGGGAGCGGCTGCTGCAGCTGGGCCAG
GGCGACGAGCTGTGCGGCCAGGAGGAGGCCTTCCGAGGCTGGGCCCAGG
CTGCCTTCCAGGCCGCCTGTGAGACCTTCTGTGTGGGAGAGGATGCCAA
GGCCGCCGCCCGAGACATCTTCAGCCCCAAACGGAGCTGGAAGCGCCAG
AGGTACCGGCTGAGCGCCCAGGCCGAGGGCCTGCAGTTGCTGCCAGGTC
TGATCCACGTGCACAGGCGGAAGATGTTCCAGGCTACAATCCGCTCAGT
GGAAAACCTGCAAAGCAGCAAGTCCACGAGGGCCACCATCCTGGTGCGC
CTGGACACCGGAGGCCAGGAGGGGCTGCAGTACCAGCCGGGGGACCACA
TAGGTGTCTGCCCGCCCAACCGGCCCGGCCTTGTGGAGGCGCTGCTGAG
CCGCGTGGAGGACCCGCCGGCGCCCACTGAGCCCGTGGCAGTAGAGCAG
CTGGAGAAGGGCAGCCCTGGTGGCCCTCCCCCCGGCTGGGTGCGGGACC
CCCGGCTGCCCCCGTGCACGCTGCGCCAGGCTCTCACCTTCTTCCTGGA
CATCACCTCCCCACCCAGCCCTCAGCTCTTGCGGCTGCTCAGCACCTTG
GCAGAAGAGCCCAGGGAACAGCAGGAGCTGGAGGCCCTCAGCCAGGATC
CCCGACGCTACGAGGAGTGGAAGTGGTTCCGCTGCCCCACGCTGCTGGA
GGTGCTGGAGCAGTTCCCGTCGGTGGCGCTGCCTGCCCCACTGCTCCTC
ACCCAGCTGCCTCTGCTCCAGCCCCGGTACTACTCAGTCAGCTCGGCAC
CCAGCACCCACCCAGGAGAGATCCACCTCACTGTAGCTGTGCTGGCATA
CAGGACTCAGGATGGGCTGGGCCCCCTGCACTATGGAGTCTGCTCCACG
TGGCTAAGCCAGCTCAAGCCCGGAGACCCTGTGCCCTGCTTCATCCGGG
GGGCTCCCTCCTTCCGGCTGCCACCCGATCCCAGCTTGCCCTGCATCCT
GGTGGGTCCAGGCACTGGCATTGCCCCCTTCCGGGGATTCTGGCAGGAG
CGGCTGCATGACATTGAGAGCAAAGGGCTGCAGCCCACTCCCATGACTT
TGGTGTTCGGCTGCCGATGCTCCCAACTTGACCATCTCTACCGCGACGA
GGTGCAGAACGCCCAGCAGCGCGGGGTGTTTGGCCGAGTCCTCACCGCC
TTCTCCCGGGAACCTGACAACCCCAAGACCTACGTGCAGGACATCCTGA
GGACGGAGCTGGCTGCGGAGGTGCACCGCGTGCTGTGCCTCGAGCGGGG
CCACATGTTTGTCTGCGGCGATGTTACCATGGCAACCAACGTCCTGCAG
ACCGTGCAGCGCATCCTGGCGACGGAGGGCGACATGGAGCTGGACGAGG
CCGGCGACGTCATCGGCGTGCTGCGGGATCAGCAACGCTACCACGAAGA
CATTTTCGGGCTCACGCTGCGCACCCAGGAGGTGACAAGCCGCATACGC
ACCCAGAGCTTTTCCTTGCAGGAGCGTCAGTTGCGGGGCGCAGTGCCCT
GGGCGTTCGACCCTCCCGGCTCAGACACCAACAGCCCCTGAGAGCCGCC
TGGCTTTCCCTTCCAGTTCCGGGAGAGCGGCTGCCCGACTCAGGTCCGC
CCGACCAGGGGAATTCGCTAGCTCGACAATCAACCTCTGGATTACAAAA
TTTGTGAAAGATTGACTGGTATTCTTAACTATGTTGCTCCTTTTACGCT
ATGTGGATACGCTGCTTTAATGCCTTTGTATCATGCTATTGCTTCCCGT
ATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGT
GTTTGCTGACGCAACCCCCACTGGTTGGGGCATTGCCACCACCTGTCAG
CTCCTTTCCGGGACTTTCGCTTTCCCCCTCCCTATTGCCACGGCGGAAC
TCATCGCCGCCTGCCTTGCCCGCTGCTGGACAGGGGCTCGGCTGTTGGG
CACTGACAATTCCGTGGTGTTGTCGGGGAAATCATCGTCCTTTCCTTGG
CTGCTCGCCTGTGTTGCCACCTGGATTCTGCGCGGGACGTCCTTCTGCT
ACGTCCCTTCGGCCCTCAATCCAGCGGACCTTCCTTCCCGCGGCCTGCT
GCCGGCTCTGCGGCCTCTTCCGCGTCTTCGCCTTCGCCCTCAGACGAGT
CGGATCTCCCTTTGGGCCGCCTCCCCGCCTGGTACCTTTAAGACCAATG
ACTTACAAGGCAGCTGTAGATCTTAGCCACTTTTTAAAAGAAAAGGGGG
GACTGGAAGGGCTAATTCACTCCCAACGAAGATAAGATCTGCTTTTTGC
TTGTACTGGGTCTCTCTGGTTAGACCAGATCTGAGCCTGGGAGCTCTCT
GGCTAACTAGGGAACCCACTGCTTAAGCCTCAATAAAGCTTGCCTTGAG
TGCTTCAAGTAGTGTGTGCCCGTCTGTTGTGTGACTCTGGTAACTAGAG
ATCCCTCAGACCCTTTTAGTCAGTGTGGAAAATCTCTAGCAGTAGTAGT
GTCATGTCATCTTATTATTCAGTATTTATAACTTGCAAAGAAATGAATA
TCAGAGAGTGAGAGGAACTTGTTTATTGCAGCTTATAATGGTTACAAAT
AAAGCAATAGCATCACAAATTTCACAAATAAAGCATTTTTTTCACTGCA
TTCTAGTTGTGTTTGTCCAAACTCATCAATGTATCTTATCATGTCTGGC
TCTAGCTATCCCGCCCCTAACTCCGCCCATCCCGCCCCTAACTCCGCCC
AGTTCCGCCCATTCTCCGCCCCATGGCTGACTAATTTTTTTTATTTATG
CAGAGGCCGAGGCCGCCTCGGCCTCTGAGCTATTCCAGAAGTAGTGAGG
AGGCTTTTTTGGAGGCCTAGACTTTTGCAGATCGACCCATGGGGGCCCG
CCCCAACTGGGGTAACCTTTG

In particular nonlimiting embodiments, the cells are transformed using a nanoplasmid comprising the truncated CMV enhancer promoter operably linked to endothelial nitric oxide synthase (eNOS) having the sequence ID NO. 4:

GGCTCCCCGGGCGCGACTAGTGAATTGATACTAGTATTATGCCCAGTAC
ATGACCTTATGGGACTTTCCTACTTGGCAGTACATCTACGTATTAGTCA
TCGCTATTACCATGGTGATGCGGTTTTGGCAGTACATCAATGGGCGTGG
ATAGCGGTTTGACTCACGGGGATTTCCAAGTCTCCACCCCATTGACGTC
AATGGGAGTTTGTTTTGGCACCAAAATCAACGGGACTTTCCAAAATGTC
GTAACAACTCCGCCCCATTGACGCAAATGGGCGGTAGGCGTGTACGGTG
GGAGGTTTATATAAGCAGAGCTCGTTTAGTGAACCGTCAGATCGCCTGG
AGACGCCATCCACGCTGTTTTGACCTCCATAGAAGATTCTAGAGTCGAC
GCCACCATGGGCAACTTGAAGAGCGTGGCCCAGGAGCCTGGGCCACCCT
GCGGCCTGGGGCTGGGGCTGGGCCTTGGGCTGTGCGGCAAGCAGGGCCC
AGCCACCCCGGCCCCTGAGCCCAGCCGGGCCCCAGCATCCCTACTCCCA
CCAGCGCCAGAACACAGCCCCCCGAGCTCCCCGCTAACCCAGCCCCCAG
AGGGGCCCAAGTTCCCTCGTGTGAAGAACTGGGAGGTGGGGAGCATCAC
CTATGACACCCTCAGCGCCCAGGCGCAGCAGGATGGGCCCTGCACCCCA
AGACGCTGCCTGGGCTCCCTGGTATTTCCACGGAAACTACAGGGCCGGC
CCTCCCCCGGCCCCCCGGCCCCTGAGCAGCTGCTGAGTCAGGCCCGGGA
CTTCATCAACCAGTACTACAGCTCCATTAAGAGGAGCGGCTCCCAGGCC
CACGAACAGCGGCTTCAAGAGGTGGAAGCCGAGGTGGCAGCCACAGGCA
CCTACCAGCTTAGGGAGAGCGAGCTGGTGTTCGGGGCTAAGCAGGCCTG
GCGCAACGCTCCCCGCTGCGTGGGCCGGATCCAGTGGGGGAAGCTGCAG
GTGTTCGATGCCCGGGACTGCAGGTCTGCACAGGAAATGTTCACCTACA
TCTGCAACCACATCAAGTATGCCACCAACCGGGGCAACCTTCGCTCGGC
CATCACAGTGTTCCCGCAGCGCTGCCCTGGCCGAGGAGACTTCCGAATC
TGGAACAGCCAGCTGGTGCGCTACGCGGGCTACCGGCAGCAGGACGGCT
CTGTGCGGGGGGACCCAGCCAACGTGGAGATCACCGAGCTCTGCATTCA
GCACGGCTGGACCCCAGGAAACGGTCGCTTCGACGTGCTGCCCCTGCTG
CTGCAGGCCCCAGATGAGCCCCCAGAACTCTTCCTTCTGCCCCCCGAGC
GTGGTCCTTGAGGTGCCCCTGGAGCACCCCACGCTGGATGGTTTGCAGC
CCTGGGCCTGCGCTGGTACGCCCTCCCGGCAGTGTCCAACATGCTGCTG
GAAATTGGGGGCCTGGAGTTCCCCGCAGCCCCCTTCAGTGGCTGGTACA
TGAGCACTGAGATCGGCACGAGGAACCTGTGTGACCCTCACCGCTACAA
CATCCTGGAGGATGTGGCTGTCTGCATGGACCTGGATACCCGGACCACC
TCGTCCCTGTGGAAAGACAAGGCAGCAGTGGAAATCAACGTGGCCGTGC
TGCACAGTTACCAGCTAGCCAAAGTCACCATCGTGGACCACCACGCCGC
CACGGCCTCTTTCATGAAGCACCTGGAGAATGAGCAGAAGGCCAGGGGG
GGCTGCCCTGCAGACTGGGCCTGGATCGTGCCCCCCATCTCGGGCAGCC
TCACTCCTGTTTTCCATCAGGAGATGGTCAACTATTTCCTGTCCCCGGC
CTTCCGCTACCAGCCAGACCCCTGGAAGGGGAGTGCCGCCAAGGGCACC
GGCATCACCAGGAAGAAGACCTTTAAAGAAGTGGCCAACGCCGTGAAGA
TCTCCGCCTCGCTCATGGGCACGGTGATGGCGAAGCGAGTGAAGGCGAC
AATCCTGTATGGCTCCGAGACCGGCCGGGCCCAGAGCTACGCACAGCAG
CTGGGGAGACTCTTCCGGAAGGCTTTTGATCCCCGGGTCCTGTGTATGG
ATGAGTATGACGTGGTGTCCCTCGAACACGAGACGCTGGTGCTGGTGGT
AACCAGCACATTTGGGAATGGGGATCCCCCGGAGAATGGAGAGAGCTTT
GCAGCTGCCCTGATGGAGATGTCCGGCCCCTACAACAGCTCCCCTCGGC
CGGAACAGCACAAGAGTTATAAGATCCGCTTCAACAGCATCTCCTGCTC
AGACCCACTGGTGTCCTCTTGGCGGCGGAAGAGGAAGGAGTCCAGTAAC
ACAGACAGTGCAGGGGCCCTGGGCACCCTCAGGTTCTGTGTGTTCGGGC
TCGGCTCCCGGGCATACCCCCACTTCTGCGCCTTTGCTCGTGCCGTGGA
CCACACGGCTGGAGGAACTGGGCGGGGAGCGGCTGCTGCAGCTGGGCCA
GGGCGACGAGCTGTGCGGCAGGAGGAGGCCTTCCGAGGCTGGGCCCAGG
CTGCCTTCCAGGCCGCCTGTGAGACCTTCTGTGTGGGAGAGGATGCCAA
GGCCGCCGCCCGAGACATCTTCAGCCCCAAACGGAGCTGGAAGCGCCAG
AGGTACCGGCTGAGCGCCCAGGCCGAGGGCCTGCAGTTGCTGCCAGGTC
TGATCCACGTGCACAGGCGGAAGATGTTCCAGGCTACAATCCGCTCAGT
GGAAAACCTGCAAAGCAGCAAGTCCACGAGGGCCACCATCCTGGTGCGC
CTGGACACCGGAGGCCAGGAGGGGCTGCAGTACCAGCCGGGGGACCACA
TAGGTGTCTGCCCGCCCAACCGGCCCGGCCTTGTGGAGGCGCTGCTGAG
CCGCGTGGAGGACCCGCCGGCGCCCACTGAGCCCGTGGCAGTAGAGCAG
CTGGAGAAGGGCAGCCCTGGTGGCCCTCCCCCCGGCTGGGTGCGGGACC
CCCGGCTGCCCCCGTGCACGCTGCGCCAGGCTCTCACCTTCTTCCTGGA
CATCACCTCCCCACCCAGCCCTCAGCTCTTGCGGCTGCTCAGCACCTTG
GCAGAAGAGCCCAGGGAACAGCAGGAGCTGGAGGCCCTCAGCCAGGATC
CCCGACGCTACGAGGAGTGGAAGTGGTTCCGCTGCCCCACGCTGCTGGA
GGTGCTGGAGCAGTTCCCGTCGGTGGCGCTGCCTGCCCCACTGCTCCTC
ACCCAGCTGCCTCTGCTCCAGCCCCGGTACTACTCAGTCAGCTCGGCAC
CCAGCACCCACCCAGGAGAGATCCACCTCACTGTAGCTGTGCTGGCATA
CAGGACTCAGGATGGGCTGGGCCCCCTGCACTATGGAGTCTGCTCCACG
TGGCTAAGCCAGCTCAAGCCCGGAGACCCTGTGCCCTGCTTCATCCGGG
GGGCTCCCTCCTTCCGGCTGCCACCCGATCCCAGCTTGCCCTGCATCCT
GGTGGGTCCAGGCACTGGCATTGCCCCCTTCCGGGGATTCTGGCAGGAG
CGGCTGCATGACATTGAGAGCAAAGGGCTGCAGCCCACTCCCATGACTT
TGGTGTTCGGCTGCCGATGCTCCCAACTTGACCATCTCTACCGCGACGA
GGTGCAGAACGCCCAGCAGCGCGGGGTGTTTGGCCGAGTCCTCACCGCC
TTCTCCCGGGAACCTGACAACCCCAAGACCTACGTGCAGGACATCCTGA
GGACGGAGCTGGCTGCGGAGGTGCACCGCGTGCTGTGCCTCGAGCGGGG
CCACATGTTTGTCTGCGGCGATGTTACCATGGCAACCAACGTCCTGCAG
ACCGTGCAGCGCATCCTGGCGACGGAGGGCGACATGGAGCTGGACGAGG
CCGGCGACGTCATCGGCGTGCTGCGGGATCAGCAACGCTACCACGAAGA
CATTTTCGGGCTCACGCTGCGCACCCAGGAGGTGACAAGCCGCATACGC
ACCCAGAGCTTTTCCTTGCAGGAGCGTCAGTTGCGGGGCGCAGTGCCCT
GGGCGTTCGACCCTCCCGGCTCAGACACCAACAGCCCCTGAGAGCCGCC
TGGCTTTCCCTTCCAGTTCCGGGAGAGCGGCTGCCCGACTCAGGTCCGC
CCGACCAGGGGAATTCGCTAGCTCGACAATCAACCTCTGGATTACAAAA
TTTGTGAAAGATTGACTGGTATTCTTAACTATGTTGCTCCTTTTACGCT
ATGTGGATACGCTGCTTTAATGCCTTTGTATCATGCTATTGCTTCCCGT
ATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGT
GTTTGCTGACGCAACCCCCACTGGTTGGGGCATTGCCACCACCTGTCAG
CTCCTTTCCGGGACTTTCGCTTTCCCCCTCCCTATTGCCACGGCGGAAC
TCATCGCCGCCTGCCTTGCCCGCTGCTGGACAGGGGCTCGGCTGTTGGG
CACTGACAATTCCGTGGTGTTGTCGGGGAAATCATCGTCCTTTCCTTGG
CTGCTCGCCTGTGTTGCCACCTGGATTCTGCGCGGGACGTCCTTCTGCT
ACGTCCCTTCGGCCCTCAATCCAGCGGACCTTCCTTCCCGCGGCCTGCT
GCCGGCTCTGCGGCCTCTTCCGCGTCTTCGCCTTCGCCCTCAGACGAGT
CGGATCTCCCTTTGGGCCGCCTCCCCGCCTGGTACCTTTAAGACCAATG
ACTTACAAGGCAGCTGTAGATCTTAGCCACTTTTTCTAGAGGGCCCGAG
ATCTTTTTCCCTCTGCCAAAAATTATGGGGACATCATGAAGCCCCTTGA
GCATCTGACTTCTGGCTAATAAAGGAAATTTATTTTCATTGCAATAGTG
TGTTGGAATTTTTTGTGTCTCTCACTCGGAAGGACATAAGGGCGGCCCT
AGCCCGCCTAATGAGCGGGCTTTTTTTTGGCTTGTTGTCCACAACCGTT
AAACCTTAAAAGCTTTAAAAGCCTTATATATTCTTTTTTTTCTTATAAA
ACTTAAAACCTTAGAGGCTATTTAAGTTGCTGATTTATATTAATTTTAT
TGTTCAAACATGAGAGCTTAGTACGTGAAACATGAGAGCTTAGTACGTT
AGCCATGAGAGCTTAGTACGTTAGCCATGAGGGTTTAGTTCGTTAAACA
TGAGAGCTTAGTACGTTAAACATGAGAGCTTAGTACGTACTATCAACAG
GTTGAACTGCTGATCCACGTTGTGGTAGAATTGGTAAAGAGAGTCGTGT
AAAATATCGAGTTCGCACATCTTGTTGTCTGATTATTGATTTTTGGCGA
AACCATTTGATCATATGACAAGATGTGTATCTACCTTAACTTAATGATT
TTGATAAAAATCATTAGGTACAAAAGAAAAGGGGGGACTGGAAGGGCTA
ATTCACTCCCAACGAAGATAAGATCTGCTTTTTGCTTGTACTGGGTCTC
TCTGGTTAGACCAGATCTGAGCCTGGGAGCTCTCTGGCTAACTAGGGAA
CCCACTGCTTAAGCCTCAATAAAGCTTGCCTTGAGTGCTTCAAGTAGTG
TGTGCCCGTCTGTTGTGTGACTCTGGTAACTAGAGATCCCTCAGACCCT
TTTAGTCAGTGTGGAAAATCTCTAGCAGTAGTAGTTCATGTCATCTTAT
TATTCAGTATTTATAACTTGCAAAGAAATGAATATCAGAGAGTGAGAGG
AACTTGTTTATTGCAGCTTATAATGGTTACAAATAAAGCAATAGCATCA
CAAATTTCACAAATAAAGCATTTTTTTCACTGCATTCTAGTTGTGGTTT
GTCCAAACTCATCAATGTATCTTATCATGTCTGGCTCTAGCTATCCCGC
CCCTAACTCCGCCCATCCCGCCCCTAACTCCGCCCAGTTCCGCCCATTC
TCCGCCCCATGGCTGACTAATTTTTTTTATTTATGCAGAGGCCGAGGCC
GCCTCGGCCTCTGAGCTATTCCAGAAGTAGTGAGGAGGCTTTTTTGGAG
GCCTAGACTTTTGCAGATCGACCCATGGGGGCCCGCCCCAACTGGGGTA
ACCTTTG

In particular, the host cells may have therapeutic potential in their own right, even without expression of the polynucleotide construct, such as bone marrow derived (mesenchymal) stem (stromal) cells (MSCs) or other cells with regenerative potential (e.g. endothelial progenitor cells (EPCs) or endothelial-like progenitor cells, adipose tissue derived mesenchymal stem cells, multipotent adult progenitor cells (MAPCs), side population (SP) cells, lung derived progenitor or stem cells, or embryonic stems cells (ESCs), among others) in which case administration of such cells even without the benefit of gene transfection may result in therapeutic effects.

Thus, according to one aspect of the present disclosure, there is provided a process of conducting gene therapy in a mammalian patient, which comprises administering to the circulation system of the patient genetically modified mammalian cells containing at least one polynucleotide construct which is capable of expressing at least one gene product in the circulation after administration thereto. In a more specific aspect of the invention, the circulation system is the pulmonary circulation system.

According to another, more specific aspect of the disclosure, there are provided genetically modified mammalian cells selected from fibroblasts, endothelial cells, smooth muscle cells, endothelial progenitor cells, endothelial-like progenitor cells, and mesenchymal stem cells, said cells containing at least one polynucleotide construct coding for a therapeutic factor.

A further aspect of the present disclosure provides the use in the preparation of a medicament for administration to a mammalian patient to alleviate symptoms of a disorder, of viable, transfected mammalian cells containing at least one expressible polynucleotide construct coding for a therapeutic factor.

Yet another aspect of the present disclosure is a process of preparing genetic modifications of mammalian cells selected from fibroblasts, endothelial cells, and progenitor cells, which comprises transfecting said mammalian cells with at least one gene coding for a therapeutic factor, to produce transfected cells capable of expressing said therapeutic factor in vivo.

The disclosure further teaches a process of preparing transformants of mammalian cells, which comprises transfecting said mammalian cells with at least one expressible polynucleotide construct coding for a therapeutic factor to produce transformed cells capable of expressing said factor in vivo.

In yet another embodiment, the present disclosure teaches a method for treating, alleviating, or inhibiting the progression of pulmonary hypertension in a mammalian patient comprising administration to the lung by injection into the pulmonary circulation of the mammalian patient suffering from the disorder, of endothelial progenitor cells or endothelial like progenitor cells, the endothelial progenitor cells or endothelial like progenitor cells transformed to express a polynucleotide construct coding for an endothelial nitric oxide synthase. The endothelial nitric oxide synthase may be human endothelial nitric oxide synthase. In one embodiment, the cells are allogenic, syngeneic, or autologous.

In one embodiment, the pulmonary hypertension is associated with scleroderma. In another embodiment, the pulmonary hypertension is associated with congenital heart disease. In another embodiment, the pulmonary hypertension is associated with lupus (SLE). In another embodiment, the pulmonary hypertension is associated or caused by idiopathic PAH.

In one embodiment, there is provided a truncated human cytomegalovirus (CMV) enhancer element comprising SEQ ID NO: 1 or a functional derivative thereof.

In one embodiment, there is provided a polynucleotide expression cassette comprising the truncated CMV promoter of claim and a transcribable polynucleotide operably linked to the truncated CMV promoter.

In one embodiment, the transcribable polynucleotide encodes for endothelial nitric oxide synthase (eNOS).

In one embodiment, there is provided a polynucleotide construct containing the expression cassette.

In one embodiment, there is provided a pharmaceutical composition comprising the polynucleotide construct.

In one embodiment, there is provided a host cell comprising the polynucleotide construct. In one embodiment, there is provided a use of the host cell to treat pulmonary or cardiac disease in the mammalian subject in need thereof.

In one embodiment, there is provided a method of preventing or treating a pulmonary or a cardiac disease in a patient in need of treatment thereof, the method comprising
contacting a patient in need of treatment thereof with transformed host cells from a subject, said host cells transformed with the polynucleotide construct.

In one embodiment, there is provided a method of directing expression of a transcribable polynucleotide comprising transforming a host cell with the polynucleotide construct and expressing the transcribable polynucleotide.

In one embodiment, there is provided a method for producing a medicament for the treatment of a pulmonary or cardiac disease in a patient in need of treatment thereof, the method comprising:
isolating host cells from a subject;
seeding the host cells onto an extracellular matrix (ECM) coated substrate;
incubating the host cells at a low O₂ concentration and about 37 degrees Celsius; and
transforming the host cells with the polynucleotide construct to produce transformed host cells for use as a medicament for the treatment of a pulmonary or cardiac disease.

### BRIEF DESCRIPTION OF THE DRAWINGS

FIG. 1 is a restriction map of a minicircle plasmid containing the truncated enhancer element in the CMV promoter (green) and the human eNOS ORF (orange);

FIG. 2 is a restriction map of a nanoplasmid containing the highly truncated CMV enhancer element (grey in the green) as part of the CMV promoter (green) and the human eNOS ORF (orange);

FIG. 3 shows the effect of eNOS protein accumulation when EPCs are transfected with different plasmids where a) is a western blot showing expression of eNOS protein; b) is a bar graph showing eNOS fold change; and c) is a bar graph showing % change of eNOS in transfected EPCs compared to eNOS isolated from 0.5 ug of HUVECs;

FIG. 4 shows the effect of two different transfection reagents (JetPEI-Macrophage versus JetOPTIMUS) on eNOS protein accumulation in EPCs where a) is a western blot showing expression of eNOS protein at varying doses of transfection reagent; b) is a bar graph showing eNOS fold change; and c) is a bar graph showing % change of eNOS in transfected EPCs compared to eNOS isolated from 0.5 ug of HUVECs;

FIG. 5 shows the effect of Superoxide dismutase (SOD) or N-gamma-nitro-L-arginine methyl ester on eNOS protein accumulation when EPCs are transfected with JetOPTIMUS 6 ug for 2 h where a) is a western blot showing expression of eNOS protein at varying doses of SOD or L-NAME; and b) is a bar graph showing eNOS fold change when EPCs are transfected with JetOPTIMUS 6 ug for 2 h;

FIG. 6. shows a schematic overview of the process of patient sample collection, harvesting, culturing and transfecting of EPCs, and the delivery of the final cell product to the patient;

FIG. 7. shows a schematic for the process for culturing and transfecting the cultured EPCs, and preparation of the final cell product; and

FIG. 8. shows a schematic for the process of administration of the final cell to the patient over multiple doses.

### DETAILED DESCRIPTION

According to the disclosure, the term “isolated” as used herein refers to an isolated nucleic acid molecule that, by the hand of man, exists outside its native environment and is therefore not a product of nature. An isolated nucleic acid molecule may exist in a purified form or in a non-native environment, such as a transgenic host cell.

As used in the application, the term “promoter” refers to nucleic acid sequences that regulate, either directly or indirectly, the transcription of corresponding nucleic acid coding sequences to which they are operably linked (e.g., a transgene or endogenous gene). In particular, the promoter refers to a DNA regulatory region capable of binding directly or indirectly to RNA polymerase and other proteins (trans-acting transcription factors) in a cell and initiating transcription of a downstream (3′ direction) coding sequence and is bound at its 3′ terminus by the transcription initiation site and extends upstream (5′ direction) to include the minimum number of bases or elements necessary to initiate transcription at levels detectable above background.

When operably linked to a transcribable polynucleotide molecule, a promoter typically causes the transcribable polynucleotide molecule to be transcribed in a manner that is similar to the transcription of the polynucleotide molecule that is normally associated with the promoter.

A promoter may function alone to regulate transcription or may act in concert with one or more other regulatory sequences (e.g., enhancers or silencers). In the context of the application, a promoter is typically operably linked to regulatory elements to regulate transcription of a transcribable gene.

As used herein, the term “transcribable polynucleotide molecule” refers to any polynucleotide molecule capable of being transcribed into a RNA molecule.

As used herein, the term “heterologous transcribable polynucleotide molecule” refers to a nucleic acid sequence not naturally associated with the host genome into which it is introduced, including non-naturally occurring multiple copies of a naturally occurring nucleic acid sequence.

As used herein, the phrase “polynucleotide construct” refers to any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecules have been linked in a functionally operative manner. The terms “polynucleotide construct” and “construct” are used interchangeably herein.

As used herein, the term “transformed” refers to a cell, tissue, organ, or organism into which a foreign polynucleotide molecule, such as a construct, has been introduced.

As used herein, the term “expression” includes any step involved in the production of the polypeptide including, but not limited to, transcription, post-transcriptional modification, translation, post-translational modification, and secretion.

As used herein, the term “nucleic acid expression cassette” refers to nucleic acid molecules that include one or more transcriptional control elements (such as, but not limited to promoters, enhancers and/or regulatory elements, polyadenylation sequences, and introns) that are operably linked to a (trans) gene encoding a polypeptide to direct expression of the (trans) gene.

The term “functional derivative” as used in the application refers to fragments of the sequences disclosed herein that retain the capability of regulating expression of the (trans) gene in the same way as the sequence from which they are derived. As used herein, the functional derivative denotes, in the context of a functional derivative of a sequence whether an nucleic acid or amino acid sequence, a molecule that retains a biological activity (either function or structural) that is substantially similar to that of the original sequence. This functional derivative or equivalent may be a natural derivative or may be prepared synthetically. The term “functional derivatives is intended to include “fragments”, “segments”, “variants” “analogs” or “chemical derivatives” of the subject matter of the present disclosure. Thus, the term “variant” refers herein to a nucleic acid molecule which is substantially similar in structure and biological activity to the nucleic acid of the present disclosure. The functional derivatives of the present disclosure can be synthesized chemically or produced through recombinant DNA technology. All these methods are well known in the art.

The term “operably linked” as used herein refers to the arrangement of various nucleic acid molecule elements relative to each such that the elements are functionally connected and are able to interact with each other. Such elements may include, without limitation, a promoter, an enhancer and/or a regulatory element, a polyadenylation sequence, one or more introns and/or exons, and a coding sequence of a gene of interest to be expressed (e.g., a transgene). The nucleic acid sequence elements, when properly oriented or operably linked, act together to modulate the activity of one another, and ultimately may affect the level of expression of the transgene. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements. Thus, two sequences, such as a promoter and a “reporter sequence” or “therapeutic sequence” are operably linked if transcription commencing in the promoter will produce an RNA transcript of the reporter sequence or therapeutic sequence. In order to be “operably linked” it is not necessary that two sequences be immediately adjacent to one another.

The term “transgene” as used herein refers to particular nucleic acid sequences encoding a polypeptide or a portion of a polypeptide to be expressed in a cell into which the nucleic acid sequence is inserted. The term “transgene” is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; and (3) a nucleic acid sequence that serves to add additional copies of the same (i.e., homologous) or a similar nucleic acid sequence naturally occurring in the cell into which it has been introduced.

As used herein, the term “expression vector” refers to a linear or circular DNA molecule that comprises a polynucleotide encoding a polypeptide of the present disclosure and is operably linked to additional nucleotides that provide for its expression.

The vector is used to transport the insert nucleic acid molecule into a suitable host cell. Once in the host cell, the vector can replicate independently of, or coincidental with, the host chromosomal DNA, and several copies of the vector and its inserted nucleic acid molecule may be generated.

According to a further particular embodiment, the vectors contain an expression cassette as described herein. The vectors can be episomal vectors (i.e., that do not integrate into the genome of a host cell), or can be vectors that integrate into the host cell genome. Examples of episomal vectors include (extrachromosomal) plasmids and so-called mini-circles, which are composed of the expression cassette only and are devoid of bacterial sequences. The smaller molecular size of minicircles enable more efficient transfections and offers sustained expression over a period of weeks as compared to standard plasmid vectors that only work for a few days.

As used herein, the term “host cell” includes any cell type that is susceptible to transformation, transfection, transduction, and the like with a nucleic acid construct or expression vector comprising a polynucleotide of the present disclosure.

Also provided by the present disclosure are host cells transformed by the polynucleotide constructs described herein. In this context, transformation refers to any process by which nucleic acid material is introduced into and expressed within a cell. Thus, transformation as used herein includes “transient” transfection procedures, including but not limited to those mediated by electroporation, cationic lipid/DNA complexes, protein/DNA complexes, calcium phosphate-mediated pinocytosis, virus vectors, etc., where a nucleic acid introduced into the host cell exists extrachromosomally.

Moreover, transformation as used herein may refer to so-called “stable” transfection methods, wherein a particular nucleic acid is introduced into a host cell in combination with a second nucleic acid encoding a selectable marker (e.g. resistance to an antibiotic), which enables the positive selection of cells in which the transfected nucleic acids have been integrated into the genome of the host cell.

The terms “inhibiting,” “reducing,” or “preventing,” or any variation of these terms, when used in the claims and/or the specification includes any measurable decrease or complete inhibition to achieve a desired result. Desired results include but are not limited to palliation, reduction, slowing, or eradication of a pulmonary or cardiovascular disease, as well as an improved quality or extension of life.

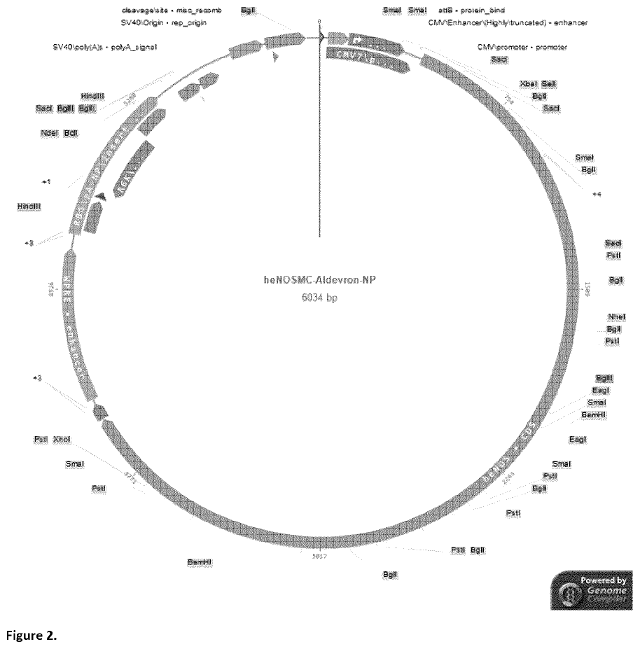

The present disclosure is based on the surprising finding that a truncated human cytomegalovirus (CMV) enhancer element as depicted schematically in FIG. 2, enhances its activity as a transcriptional regulator.

In particular nonlimiting embodiments, the truncated CMV enhancer element of the present disclosure has the nucleic acid sequence ID NO. 1.

In particular nonlimiting embodiments, the truncated CMV enhancer element is a functional fragment having a sequence identity of from 80%, 85%, 90%, or 95% sequence identity to the sequence ID NO. 1.

In particular nonlimiting embodiments, there is provided a truncated CMV promoter with the truncated CMV enhancer element of the present disclosure having the sequence ID NO. 2.

The present disclosure provides methods for treating genetic, metabolic or acquired diseases. In a nonlimiting embodiment, the present disclosure provides a method for expressing a nucleic acid molecule of the invention in a cell, the method comprising contacting the cell with a sufficient amount of a nucleic acid molecule and/or polynucleotide construct of the present disclosure. Preferably, the method is performed under conditions in which the transgene of interest is expressed in the cell.

In another nonlimiting embodiment, the present disclosure provides a method for treating a subject having, or at risk of having, pulmonary or myocardial disease, the method comprising contacting the subject in need of such treatment with a sufficient amount of a nucleic acid molecule, vector, and/or cell of the present disclosure.

In particular nonlimiting embodiments, the present disclosure provides methods for treating a subject with pulmonary hypertension. The method may comprise contacting the subject in need of such treatment with a sufficient amount of a construct, wherein the construct comprises a CMV promoter with the truncated CMV enhancer element having the nucleic acid sequence of SEQ ID NO: 1, wherein the nucleic acid is operably linked to a transgene encoding eNOS.

In particular nonlimiting embodiments, the present disclosure provides a use of a sufficient amount of a construct, for treating a subject with pulmonary hypertension, wherein the construct comprises a CMV promoter with the truncated CMV enhancer element having the nucleic acid sequence of SEQ ID NO: 1, wherein the nucleic acid is operably linked to a transgene encoding eNOS.

In particular nonlimiting embodiments, the present disclosure provides a use of a sufficient amount of a construct, for treating a subject with cardiovascular disease, wherein the construct comprises a CMV promoter with the truncated CMV enhancer element having the nucleic acid sequence of SEQ ID NO: 1, wherein the nucleic acid is operably linked to a transgene encoding eNOS.

In particular nonlimiting embodiments, the present disclosure provides methods for preparing a medicament for treating a subject having, or at risk of having, pulmonary or cardiovascular disease, the method as follows.

Human late outgrowth endothelial progenitor cells (EPCs) exhibit high proliferation capacity, contribute to neovascularization, and participate in re-endothelialization of damaged or denuded surfaces. Endothelial nitric oxide synthase (eNOS) catalyzes the production of nitric oxide, and is involved in regulation of vessel tone and angiogenesis in inflammation and ischemic cardiovascular diseases. Restoring endothelial functional activity ameliorates, treats, or prevents pulmonary or cardiovascular disease. Current applications of plasmid-based gene therapy are limited by inefficient transgene expression and adverse responses to bacterial motifs.

The following examples are intended for illustration purposes only, and should not be construed as limiting the scope of the invention in any way.

### Example 1—Novel Promoter with Truncated Enhancer to Increase Expression of Endothelial Nitric Oxide Synthase (eNOS) Protein in Transfected Endothelial Progenitor Cells (EPCs)

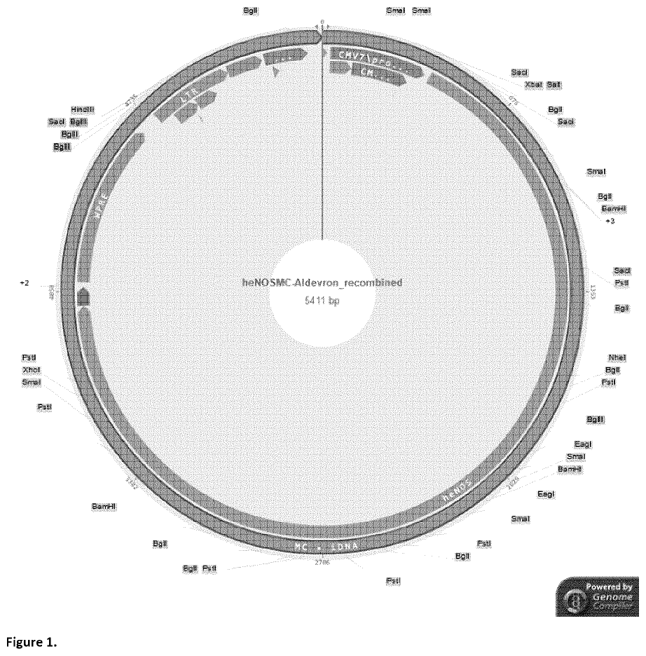

We have identified a novel promoter sequence with a truncated CMV enhancer element that was generated via homologous recombination during the production of a minicircle plasmid from a parental plasmid (FIG. 1).

The truncated CMV promoter sequence was cloned into a nanoplasmid vector along with endothelial nitric oxide synthase (eNOS) open reading frame (ORF) (FIG. 2).

### Example 2—eNOS Protein Accumulation in EPC

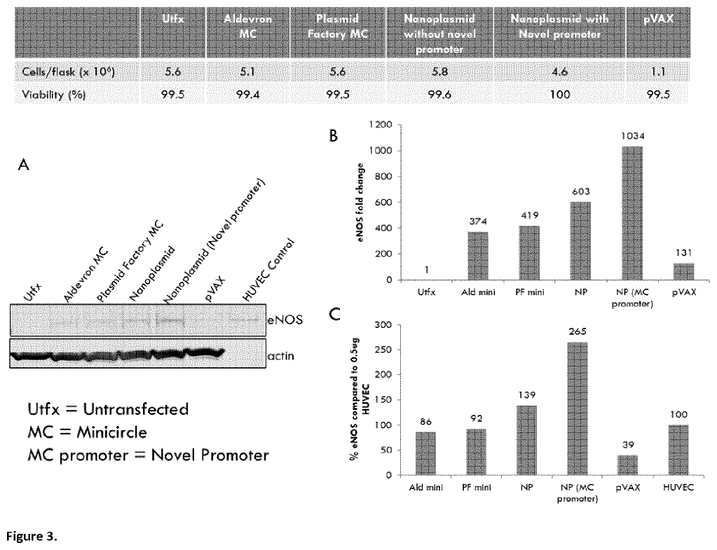

As shown in FIGS. 3A and 3B, there was enhanced eNOS protein accumulation in EPCs transfected with nanoplasmid containing the novel promoter (NP (MC promoter)) in comparison to original nanoplasmid with stock CMV promoter (NP) (FIGS. 3a, 3b, and 3c).

As shown in FIGS. 3A and 3B, there was enhanced accumulation of eNOS protein in cells transfected with minicircle containing the CMV promoter including the truncated CMV enhancer element (Aldevron mini (Aldevron, LLC, Germany) and PlasmidFactory mini (PlasmidFactory, Fargo, ND)) when compared to pVax (which is the control plasmis with full length CMV promoter and eNOS ORF). As well, there was enhanced accumulation of eNOS protein in cells transfected with nanoplasmid containing the CMV promoter including the truncated CMV enhancer element (NP (MC promoter)) as compared to NP (which is the nanoplasmid with control full length CMV promoter and eNOS ORF). As can be seen in FIG. 3C the amount of eNOS accumulation is similar to levels of eNOS observed in 0.5 micrograms of human umbilical vein endothelial (HUVEC) lysate which is internal control (FIG. 3C). When compared to pVAX, there is a significant increase in expression using mincircle and nanoplasmid with an observed about 7-fold increase with the nanoplasmid with the CMV promoter including the truncated CMV enhancer element.

Taken together, the results show significantly enhanced eNOS protein accumulation in EPCs transfected with nanoplasmid and minicircle containing the truncated CMV promoter in comparison to plasmids containing the full length CMV promoter.

### Example 3—eNOS Protein Accumulation in EPC Transfected Using Minicircle Vector Containing the Truncated CMV Promoter Construct and eNOS

Lysates from EPCs transfected with noted constructs or controls (of FIG. 1) were harvested and eNOS protein expression as compared with HUVECs were verified using ELISA.

TABLE 1

| | pg of eNOS | |
|---|---|---|
| | protein per 100 ug | |
| Samples | of cell lysate | Notes |
| Untransfected Cells | 0 | Below Detectable limit |
| pVax Transfected Cells | 8.7 | |
| Minicircle Transfected | 23.4 | |
| Cells* | | |
| Nanoplasmid | 29.5 | |
| Nanoplasmid with | 42.5 | |
| Minicircle promoter | | |
| HUVECs | 802 | 80.2 pg/10 ug of |
| | | HUVEC lysate |

*from 3 repeats

### Example 4—Specific Transfection Reagents Enhanced eNOS Protein Accumulation in EPCs

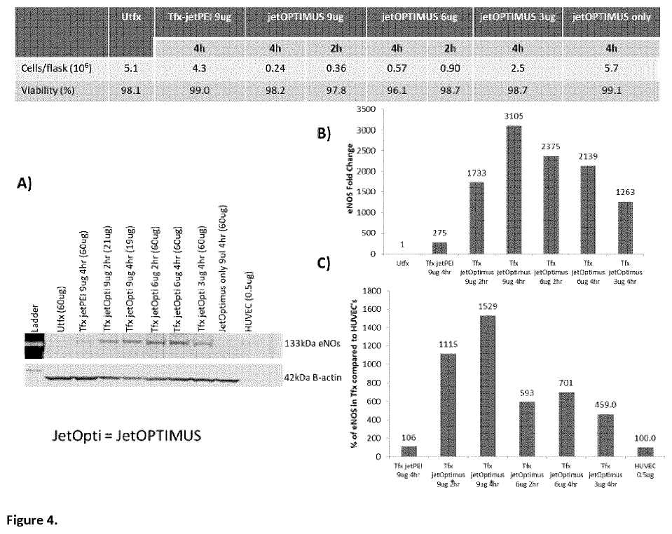

To further increase eNOS accumulation in EPCs, we transfected EPCs with multiple transfection reagents. Ten-fold higher accumulation of eNOS was observed when cells were transfected with minicircle containing the truncated CMV promoter using the JetOPTIMUS reagent when compared to the JetPEI-Macrophage reagent (FIG. 4).

Significantly, even a reduction in the amount of eNOS minicircle plasmid used for transfection with JetOPTIMUS (3 or 6 ug of eNOS minicircle DNA) still resulted in enhanced expression as compared to JetPEI (9 ug of eNOS minicircle DNA) (FIGS. 4a to 4c).

### Example 5—Transformed Cell Viability is Enhanced with SOD

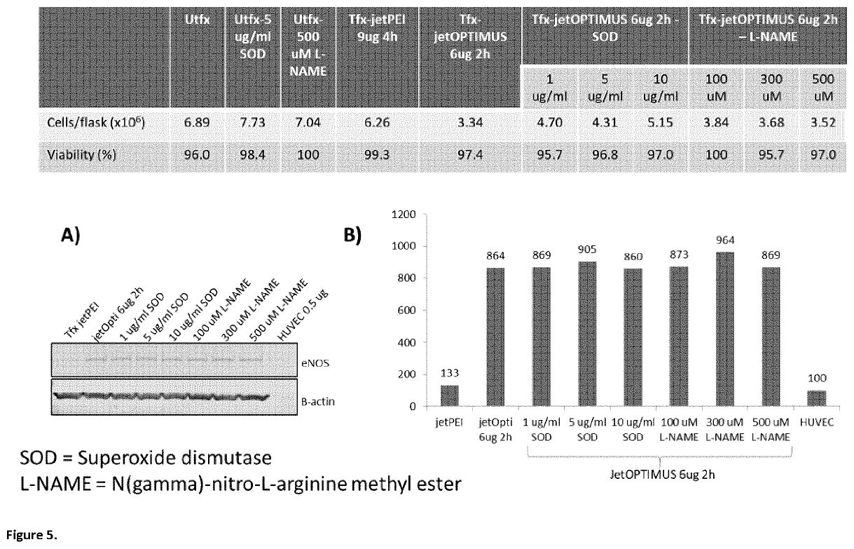

To determine whether an observed slight increase in cell death it due to the formation JetOPTIMUS-eNOS complexes or the production of eNOS, we employed the use of superoxide dismutase (SOD) to reduce free radical or N (gamma)-nitro-L-arginine methyl ester (L-NAME) in culture after transfection with eNOS using the JetOPTIMUS reagent. As shown in FIG. 5, dose-dependent treatment with SOD resulted in enhanced cell yields while maintaining eNOS protein accumulation in cells transfected using JetOPTMUS. In comparison, L-NAME had no impact on increasing cell yields when compared to cells transfected with JetOPTIMUS alone.

### Example 6—Harvesting and Growing Monocytes and Transfecting EPCs

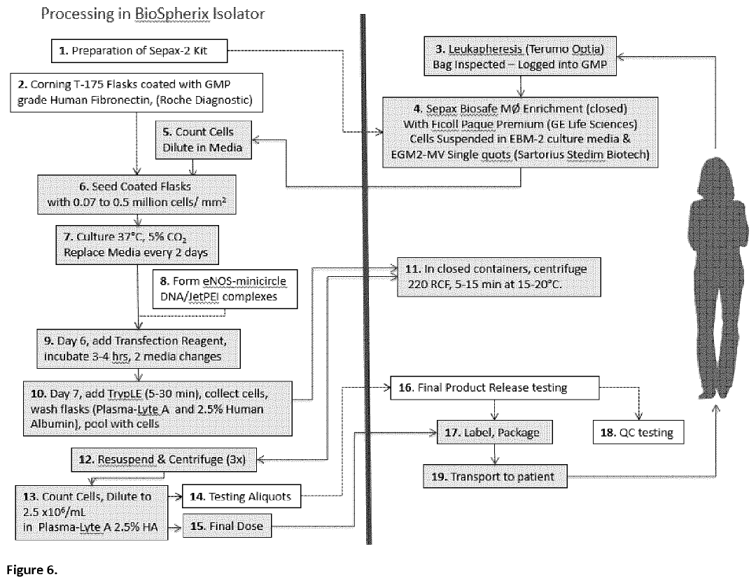

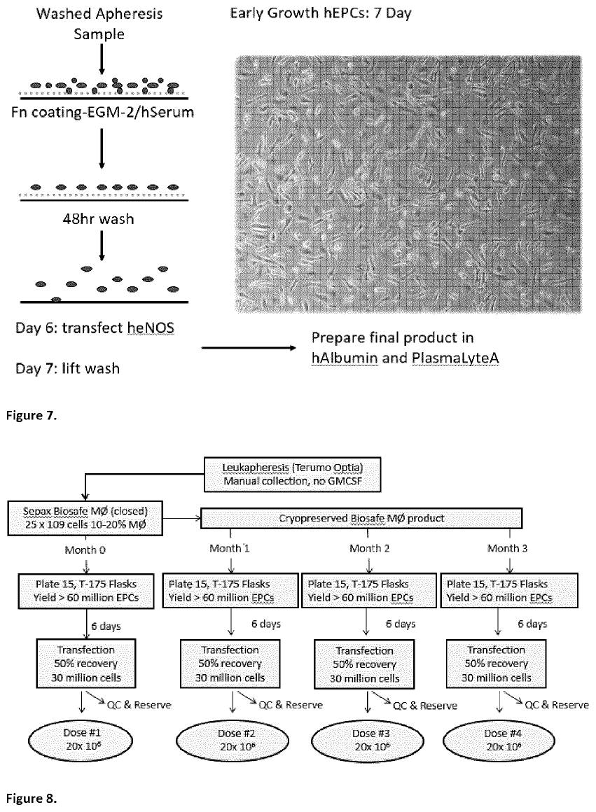

FIG. 6 shows an overview of the process of harvesting, and growing monocytes and transfecting EPCs for use in treatment.

All manufacturing processes are performed within the cell manufacturing center at the Ottawa Hospital Research Institute according to a detailed series of SOPs. Cell products are spatially temporally separated during processing in ISO8 class rooms. All open manufacturing steps are performed in an environmentally controlled Biospherix isolator operating at ISO5 or better.

Consenting patients who meet eligibility criteria undergo leukapheresis to collect peripheral blood MNCs and plasma before randomization.

Using the collected blood, MNCs are harvested using the Sepax-2 system (BioSafe, 61893122) with Ficoll-Paque Premium (GE Life Sciences) or by any conventional Ficoll centrifugation (e.g. Ficoll-Paque Premium). The enriched MNCs are counted, diluted to about 0.5-2.5×10⁶ cells/mL.

The mononuclear cells are re-suspended in complete growth medium (CGM), counted and titrated to a density of 0.5-2.5 million/mL with additional CGM.

CGM is prepared from EBM media (Endothelial Basal Media (Provitro, 262-1101) by adding basal media supplements: Hydrocortisone, recombinant human Endothelial Growth Factor, recombinant human Vascular Endothelial Growth Factor, recombinant human Insulin-like Growth Factor 1, recombinant Basic Fibroblast Growth Factor, Ascorbic Acid, Gentamycin, and Amphotericin B), and then pooled human serum is added to a final concentration of 20% Volume/Volume, and the complete growth medium (CGM) is stored at 4° C. until needed.

The mononuclear cell suspension is added to human fibronectin pre-coated flasks at about 0.14-0.2 mL/cm² surface area (or about 0.07 to 0.5 million cells/mm²). Pre-coated flasks are prepared by coating with human fibronectin 10 μg/mL for about 45 minutes and washing with PBS.

Alternatively, the mononuclear cells can be cryopreserved by resuspending in cryoprotectant Cryostor CS10 (BioLife Solutions/StemCell Technologies) and cryopreserved by step down freezing (e.g. −1 to −5° C. per minute up to −80° C. and then at −10° C. per minute up to −150° C.) and stored in the liquid Nitrogen phase of dedicated monitored freezer in the Cell Manufacturing Centre.

As shown in FIG. 6, the seeded flasks are placed in an incubator at about 37 degrees Celsius and about 5% CO₂. On day 2 post-plating, the complete growth media (CGM) is replaced with fresh CGM at a volume of 0.14-0.2 mL/cm² surface area. The media is replaced with CGM every 48 hours.

Cultured EPCs are examined microscopically to assure adequate cell coverage where ≥50% of culture surface area covered by attached cells with the presence of elongated and spindle-like shape cells, an absence of multi-nucleated cells or pleiotropic morphology, an absence of visible evidence of contamination, ≥70% cell viability of cells, and within acceptance limit of endotoxin levels (<5 EU/dose as measured by Endosafe PTS).

On day 5 or 6 or about 24 hours from cell harvest, mini-circle eNOS DNA and JET-PEI™ cationic lipid transfection reagent (Polyplus Transfection, cat. 300-28) are incubated for about 20 to 25 minutes to form eNOS DNA/JET-PEI complexes. The formed complexes (9 ug of DNA (1 mg/mL) in 18 μL of 7.5 mM JET-PEI) are added to the cultured EPCs per flask and the mixture is incubated under gentle rocking for a transfection incubation period about 3 to 4 hours at 37 degrees Celsius, 5% CO₂. At the end of the transfection incubation period, the flasks are washed with 0.05-0.2 ml/cm² surface area using EBM media and then replaced with 0.10-0.2 mL/cm² surface area of pre-warmed CGM.

In another embodiment, the transfection step was also performed using the JetOptimus™ transfection reagent (Polyplus Transfection, France). In particular, 3-9 ug of DNA (1 mg/mL) was diluted in 300-900 uL of jetOPTIMUS buffer and then mixed with jetOPTIMUS solution in 1:1 ratio (1 ug DNA:1 μL of JetOPTIMUS) and incubated for 10 minutes at room temperature to form eNOS/JetOPTMIUS complexes. After 10 minutes incubation, the complexes were added to each flask, as described above.

FIG. 6 shows the cultured early growth EPCs at day 7. On day 7, or about 24 hours from the end of the transfection incubation period, the flasks containing the transformed EPCS are treated with TrypLE (0.05-1 mL/cm² for about 5-30 min, preferably 5-20 min with agitation) (TrypLE Select, Invitrogen) to detach the cells. The detached cells are then washed with pre-warmed plasma-Lyte A (injection solution, Baxter) and 2.5% Human Albumin solution, with repeated washings as necessary, and the collected cells are pooled. The pooled cells are centrifuged at about 200-220 RCF for about 5 to 15 minutes at 15 to 20 degrees Celsius. The supernatant is discarded and the pellet is resuspended with the plasma-Lyte A and 2.5% Human Albumin solution, with straining through a 70 micron filter as necessary, to avoid cell clumping when clumping persists. The centrifugation and resuspension steps in plasma-Lyte A and 2.5% Human Albumin are then repeated two more times with cell counting by a hemocytometer and cell viability determination steps performed between centrifugation and resuspension steps. The final concentration is adjusted to about 2.5 million cells/mL by addition of Plasma Lyte A with 2.5% hAlb.

The placebo product is prepared on the day of delivery by diluting Human Albumin (hAlb; 25% solution) (obtained from Hospital Blood Bank) 1:10 (Volume/Volume) in Plasma Lyte A under aseptic conditions and then 8 mL of the final placebo product is transferred into a sterile 10 ml syringe, capped, labeled, and sealed in a sterile plastic bag for transport to the clinical site.

### Example 7—Delivery of Cellular Products and Cell Therapy Treatment of Patients with Refractory Hypertension

The overview of treatment of patients with refractory hypertension is shown in FIG. 8. Prior to administration, the final cellular products are viewed under light microscopic to ensure morphology of adherent cells, cell count and viability is confirmed and endotoxin levels are within acceptable levels and there is absence of contamination.

Treatment delivery takes place in a medically supervised outpatient environment that is equipped for repeated assessments of vital signs and oximetry. BP, HR, oximetry measurements of participants are taken for at least 1 hour post treatment.

For each treatment, a total of about 20 million cells or an identical volume of Plasma Lyte (8 ml) will be delivered via peripheral IV cannula at a rate of no more than 2 ml/minute.

As shown in FIG. 8, one treatment regimen includes 4 separate doses of the cellular products with each dose given within one month of a previous dose.

To confirm benefit of treatment, periodic 6 MWD assessments are conducted and the patient's hemodynamic parameters right ventricle (RV) function and lung perfusion can be assessed by Magnetic Resonance Imaging (MRI). Estimated RVSP and measure of RV function can done by echocardiography.

### Example 8—Delivery of Cellular Products and Cell Therapy Treatment for Acute Myocardial Infarction

Recovery of cardiac function in myocardial infarction (MI) patients is often modest or even absent despite widespread use of pharmacological and/or interventional reperfusion therapies.

Circulating mononuclear cells (MNCs) are obtained by apheresis as provided in Example 7 and can then subjected to differential culture for 3 days to select a population of highly regenerative, endothelial-like, culture modified MNCs (E-CMMs) (also referred herein as “early EPCs”).

Patients randomized to intervention arms will receive about 20 million autologous E-CMMs (eNOS-transfected or non-transfected). A total of 8 mL of cells or placebo is gently injected manually by the interventional cardiologist at no more than 1 mL per 45 seconds directly into the infarct-related artery distal to an inflated angioplasty balloon.

The full cell infusion is administered during balloon inflations of 90 seconds, each interrupted by 3 minutes of reflow with the balloon deflated.

The full cell infusion is administered during balloon inflations of 90 seconds, each interrupted by 3 minutes of reflow with the balloon deflated.

While the transcribable polynucleotide encoding the therapeutic factor eNOS is contemplated in the present disclosure, other therapeutic factors are within the scope of this invention. For example, therapeutic factors expressed in the lung by the transgenes released into and delivered by the circulation of other body organs downstream of the lungs are within the scope of this invention. Transgenes expressing therapeutic factors such as Factor VIII for treatment of classical haemophelia, and other clotting factors for treating various bleeding disorders may be used. Other examples include: transgenes expressing hormones, for example growth hormone for treatment of hypopituitary dysfunction, insulin, (thyroid stimulating hormone (TSH) for treatment hypothyroidism following pituitary failure, and other hormones; transgenes expressing beneficial lipoproteins such as Apo A1 and other proteins/enzymes participating in lipid metabolism such as lipoprotein lipase; transgenes expressing prostacyclin synthase or other transgenes that produce vasoactive substances; transgenes expressing anti-oxidants and free radical scavengers; transgenes expressing soluble cytokine receptors to neutralize actions of damaging levels of immune mediators, for example soluble TNFγ receptor, or cytokine receptor antagonists, for example IL1 ra; transgenes expressing soluble adhesion molecules, for example ICAM-1, to interrupt pathological cell adhesion processes such as those which occur in inflammatory diseases; transgenes expressing soluble receptors for viruses to inhibit infection of cells, e.g. CD4, CXCR4, CCR5 for HIV; transgenes expressing cytokines, for example IL-2, to activate immune responses for-combatting infections; and the cystic fibrosis gene, as a transgene.

Other examples of transgenes for use in the cell based therapy of the invention include transgenes encoding for:
elastase inhibitors for use in treating pulmonary vascular disease such as pulmonary hypertension or systemic vascular disease;
tissue inhibiting metaloproteins for use in treating atherosclerosis or arterial aneurysms;
potassium channels or potassium channel modulators for use in treating pulmonary hypertension;
anti-oxidants such as superoxide dismutase for use in treating pulmonary hypertension, ARDS and pulmonary fibrosis; and
anti-inflammatory factors such as cytokines, IL-10 and IL-4 for use in treating inflammatory vascular disease such as atherosclerosis or arterial aneurysms.

In some embodiments, the transcribable polynucleotide is CFTR, PGIS, Ang-1, vascular endothelial growth factor family (VEGF A, B, C, PIGF), fibroblast growth factor, erythropoietin, hemoxygenase-1 (HO-1) or hemoxygenase-2 (HO-2), transforming growth factor beta (or other member of the TGF-beta super family including BMPs 1, 2, 4, 7 and their receptors BMPR2 or BMPR1) or platelet derived growth factors (A or B).

In some other embodiments, the transcribable polynucleotide vascular is endothelial growth factor (VEGF) and its isoforms, fibroblast growth factor (FGF, acid and basic), angiopoietin-1 and other angiopoietins, erythropoietin, hemoxygenase, transforming growth factor-α (TGF-α), transforming growth factor-β (TGF-β) or other members of the TGF-β super family including BMPs 1, 2, 4, 7 and their receptors MBPR2 or MBPR1, hepatic growth factor (scatter factor), or hypoxia inducible factor (HIF).

In some other embodiments, the transcribable polynucleotide vascular is prostaglandin I synthase or Krupple-like factors (KLF-2, 4, and others) artificially engineered transcription factors.

The embodiments of the present application described above are intended to be examples only. Those of skill in the art may effect alterations, modifications and variations to the particular embodiments without departing from the intended scope of the present application. In particular, features from one or more of the above-described embodiments may be selected to create alternate embodiments comprised of a subcombination of features which may not be explicitly described above. In addition, features from one or more of the above-described embodiments may be selected and combined to create alternate embodiments comprised of a combination of features which may not be explicitly described above. Features suitable for such combinations and subcombinations would be readily apparent to persons skilled in the art upon review of the present application as a whole. Any dimensions provided in the drawings are provided for illustrative purposes only and are not intended to be limiting on the scope of the invention. The subject matter described herein and in the recited claims intends to cover and embrace all suitable changes in technology.

1. A truncated human cytomegalovirus (CMV) enhancer element consisting of SEQ ID NO: 1.

2. (canceled)

3. A polynucleotide expression cassette comprising the truncated CMV promoter of claim 1 and a transcribable polynucleotide which encodes for endothelial nitric oxide synthase (eNOS) operably linked to the truncated CMV promoter.

4. (canceled)

5. A polynucleotide construct containing the expression cassette of claim 3, wherein the polynucleotide construct is one or more of a minicircles, a nanoplasmid, a mini intronic plasmid, a minimalistic immunologically defined gene expression vector, a dbDNA, a SEQ ID NO: 3, and a SEQ ID NO: 4.

6. (canceled)

7. A pharmaceutical composition comprising the polynucleotide construct of claim 5.

8. A host cell comprising the polynucleotide construct of claim 5.

9-25. (canceled)

26. A method of preventing or treating a renal, vascular, pulmonary, or cardiac disease in a patient in need of treatment thereof, the method comprising contacting a patient in need of treatment thereof with transformed host cells of claim 8.

27. The method of claim 26 wherein the subject is the patient in need of treatment thereof or the subject is different from the patient in need of treatment thereof.

28. The method of claim 26 wherein the contacting is into the circulatory system of the patient.

29. The method of claim 28 wherein access into the circulatory system is through the pulmonary system.

30. The method of claim 26 wherein the pulmonary disease is pulmonary hypertension.

31. The method of claim 26 wherein the contacting is by coronary injection into an infarct-related artery to treat the cardiac disease.

32. The method of claim 26 wherein the cardiac disease is ischemic heart disease.

33. (canceled)

34. A method for producing a medicament for the treatment of a pulmonary or cardiac disease in a patient in need of treatment thereof, the method comprising:
isolating host cells from a subject;
seeding the host cells onto an extracellular matrix (ECM) coated substrate;
incubating the host cells at a low O₂ concentration and about 37 degrees Celsius; and
transforming the host cells with the polynucleotide construct of claim 5 to produce transformed host cells for use as a medicament for the treatment of a pulmonary or cardiac disease.

35. (canceled)

36. (canceled)

37. (canceled)

38. The method of claim 34 wherein the incubating is over a duration of about 3 days to about 9 days.

39. The method of claim 38 wherein the incubating is over a duration of about 6 days to about 7 days.

40. The method of claim 39 wherein the incubating is over a duration of 6 days.

41. (canceled)

42. The method of claim 34 wherein the ECM is a fibronectin.

43. (canceled)

44. (canceled)

45. (canceled)

46. The method of claim 34 wherein the host cells are mononuclear cells (MNCs).

47. The method of claim 46 wherein the incubating differentiates the MNCs into endothelial progenitor cells (EPCs).

48. (canceled)

49. The method of claim 34 wherein the isolating comprises separating marrow derived (mesenchymal) stem (stromal) cells (MSCs) from bone marrow obtained from the subject.

50.-53. (canceled)
